(19) United States
(12) Patent Application Publication
(10) Pub. No.: US 2009/0062304 A1
(43) Pub. Date: Mar. 5, 2009

(54) Benzimidazole derivatives and their use for modulating the gaba-alpha receptor complex

(76) Inventors: Lene Teuber, Vaerlose (DK); Janus S. Larsen, Holbaek (DK); Philip K. Ahring, Bagsvaerd (DK); Elsebet Ostergaard Nielsen, Kobenhavn (DK); Naheed Mirza, Birkerod (DK)

Correspondence Address: BIRCH STEWART KOLASCH & BIRCH, PO BOX 747, FALLS CHURCH, VA, 22040-0747, US

(21) Appl. No.: 11/918,681

(22) PCT Filed: Apr. 18, 2006
(86) PCT No.: PCT/EP2006/061621

§ 371 Date: Oct. 17, 2007

(30) Foreign Application Priority Data
Sep. 19, 2005 (DK) PA 2005 00566

(51) Int. Cl. C07D 403/10; A61K 31/496; A61P 25/00; A61P 25/18; A61P 25/30; A61P 25/28; A61P 25/06

(52) U.S. Cl. 514/254.06; 544/370

(57) ABSTRACT

This invention relates to novel benzimidazole derivatives, pharmaceutical compositions containing these compounds, and methods of treatment therewith.

The compounds of the invention are useful in the treatment of central nervous system diseases and disorders, which are responsive to modulation of the GABA_{A} receptor complex, and in particular for combating anxiety and related diseases.

### TECHNICAL FIELD

This invention relates to novel benzimidazole derivatives, pharmaceutical compositions containing these compounds, and methods of treatment therewith.

The compounds of the invention are useful in the treatment of central nervous system diseases and disorders, which are responsive to modulation of the GABA_{A} receptor complex, and in particular for combating anxiety and related diseases.

### BACKGROUND ART

The modulatory sites on the GABA_{A} receptor complex, such as for example the benzodiazepine binding site, are the targets for anxiolytic drugs, such as the classical anxiolytic benzodiazepines. However, they are associated with a number of undesirable features.

Multiple isoforms of the GABA_{A} receptor exist; each receptor is a pentameric complex comprising subunits drawn from α₁₋₆, β₁₋₃, γ₁₋₃, δ, ε, and θ subunit isoforms. The classical anxiolytic benzodiazepines show no subtype selectivity. It has been suggested that one of the key elements in the disadvantages of the classical benzodiazepanes (such as sedation, dependency, and cognitive impairment) is related to the α1 subunit of the GABA_{A} receptors. Thus compounds with selectivity for the α2 and/or α3 subunits over the α1 subunit are expected to have an improved side effect profile.

Thus, there is still a strong need for compounds with an optimised pharmacological profile. Furthermore, there is a strong need to find effective compounds without unwanted side effects associated with older compounds.

### SUMMARY OF THE INVENTION

In its first aspect, the invention provides a compound of Formula I:

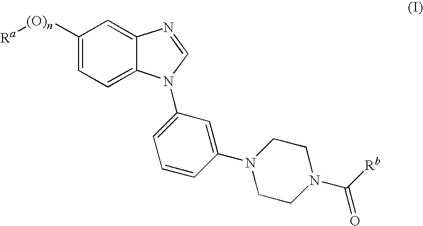

or an N-oxide thereof, any of its isomers or any mixture of its isomers, or a pharmaceutically acceptable salt thereof,
wherein R^{a}, n and R^{b} are defined as below.

In its second aspect, the invention provides a pharmaceutical composition, comprising a therapeutically effective amount of a compound of the invention, or an N-oxide thereof, any of its isomers or any mixture of its isomers, or a pharmaceutically acceptable salt thereof, together with at least one pharmaceutically acceptable carrier, excipient or diluent.

In a further aspect, the invention provides the use of a compound of the invention, or an N-oxide thereof, any of its isomers or any mixture of its isomers, or a pharmaceutically acceptable salt thereof, for the manufacture of a pharmaceutical composition for the treatment, prevention or alleviation of a disease or a disorder or a condition of a mammal, including a human, which disease, disorder or condition is responsive to modulation of the GABA_{A} receptor complex in the central nervous system.

In a still further aspect, the invention relates to a method for treatment, prevention or alleviation of a disease or a disorder or a condition of a living animal body, including a human, which disorder, disease or condition is responsive to modulation of the GABA_{A} receptor complex in the central nervous system, which method comprises the step of administering to such a living animal body in need thereof a therapeutically effective amount of a compound of the invention, or an N-oxide thereof, any of its isomers or any mixture of its isomers, or a pharmaceutically acceptable salt thereof.

Other objects of the invention will be apparent to the person skilled in the art from the following detailed description and examples.

### DETAILED DISCLOSURE OF THE INVENTION

### Substituted Benzimidazole Derivatives

In its first aspect the present invention provides a compound of the general formula (I):

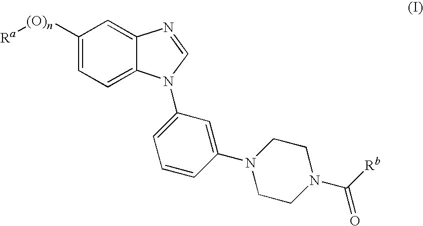

or an N-oxide thereof, any of its isomers or any mixture of its isomers,
or a pharmaceutically acceptable salt thereof,
wherein
R^{a} represents an alkyl group;
which alkyl group is optionally substituted with one or more substituents independently selected from the group consisting of:
   halo, hydroxy, R′R″N—, cyano, nitro, cycloalkyl, alkenyl, alkynyl, alkoxy, alkenyloxy, alkynyloxy, formyl, alkylcarbonyl, alkoxycarbonyl, alkoxyalkylcarbonyl or R′R″N-carbonyl-;
      wherein R′ and R″ independent of each other are hydrogen or alkyl;
         n is 0 or 1;
         R^{b} represents hydrogen or alkyl.

In one embodiment, R^{a} represents an alkyl group; which alkyl group is optionally substituted with one or more substituents independently selected from the group consisting of: halo, hydroxy, R′R″N—, cyano, nitro, cycloalkyl, alkenyl, alkynyl, alkoxy, alkenyloxy, formyl, alkylcarbonyl, alkoxycarbonyl, alkoxyalkylcarbonyl or R′R″N-carbonyl-; wherein R′ and R″ independent of each other are hydrogen or alkyl.

In a second embodiment, R^{a} represents alkyl. In a special embodiment, R^{a} represents methyl or isopropyl.

In a further embodiment, R^{a} represents alkyl substituted with one or more halo, such as perhaloalkyl. In a special embodiment, R^{a} represents trifluoromethyl.

In a still further embodiment, R^{a} represents alkyl substituted with cyano, hydroxyl, alkoxy, alkenyloxy, alkynyloxy, alkoxycarbonyl, or R′R″N-carbonyl-. In a further embodiment, R^{a} represents alkyl substituted with cyano, hydroxyl, alkoxy, alkenyloxy, alkoxycarbonyl, or R′R″N-carbonyl-.

In a further embodiment, R^{a} represents cyanoalkyl, such as cyanomethyl.

In a still further embodiment, R^{a} represents hydroxyalkyl, such as 1-hydroxyethyl.

In a further embodiment, R^{a} represents alkyl substituted with alkoxy, such as 1-alkoxyethyl. In a special embodiment, R^{a} represents methoxyalkyl, such as methoxyethyl, such as 1-methoxyethyl. In a further embodiment, R^{a} represents ethoxyalkyl, such as ethoxyethyl, such as 1-ethoxyethyl. In a still further embodiment, R^{a} represents propoxyalkyl, such as propoxyethyl, such as 1-propoxyethyl, in particular 1-isopropoxyethyl.

In a further embodiment, R^{a} represents alkyl substituted with alkynyloxy, such as prop-2-ynyloxy. In a special embodiment, R^{a} represents 1-prop-2-ynyloxy-ethyl.

In a still further embodiment, R^{a} represents alkyl substituted with alkoxycarbonyl, such as methoxycarbonylalkyl. In a special embodiment, R^{a} represents methoxycarbonylmethyl.

In a further embodiment, R^{a} represents alkyl substituted with R′R″N-carbonyl-. In a special embodiment, R′ represents hydrogen or methyl. In a further embodiment, R″ represents hydrogen or methyl. In a still further embodiment, R^{a} represents aminocarbonylmethyl, methylaminocarbonylmethyl or dimethylaminocarbonylmethyl.

In a still further embodiment, n is 0. In a still further embodiment, n is 1.

In a further embodiment, R^{b} represents alkyl, such as methyl.

In a special embodiment the chemical compound of the invention is
1-{4-[3-(5-Methyl-benzoimidazol-1-yl)-phenyl]-piperazin-1-yl}-ethanone;
1-{4-[3-(5-Trifluoromethyl-benzoimidazol-1-yl)-phenyl]-piperazin-1-yl}-ethanone;
1-{4-[3-(5-Methoxy-benzoimidazol-1-yl)-phenyl]-piperazin-1-yl}-ethanone;
1-{4-[3-(5-Trifluoromethoxy-benzoimidazol-1-yl)-phenyl]-piperazin-1-yl}-ethanone;
{1-[3-(4-Acetyl-piperazin-1-yl)-phenyl]-1H-benzoimidazol-5-yl}-acetic acid methyl ester;
{1-[3-(4-Acetyl-piperazin-1-yl)-phenyl]-1H-benzoimidazol-5-yl}-acetonitrile;
2-{1-[3-(4-Acetyl-piperazin-1-yl)-phenyl]-1H-benzoimidazol-5-yl}-acetamide;
2-{1-[3-(4-Acetyl-piperazin-1-yl)-phenyl]-1H-benzoimidazol-5-yl}-N-methyl-acetamide;
2-{1-[3-(4-Acetyl-piperazin-1-yl)-phenyl]-1H-benzoimidazol-5-yl}-N,N-dimethyl-acetamide;
1-(4-{3-[5-(1-Hydroxy-ethyl)-benzoimidazol-1-yl]-phenyl}-piperazin-1-yl)-ethanone;
1-(4-{3-[5-(1-Methoxy-ethyl)-benzoimidazol-1-yl]-phenyl}-piperazin-1-yl)-ethanone;
1-(4-{3-[5-(1-Prop-2-ynyloxy-ethyl)-benzoimidazol-1-yl]-phenyl}-piperazin-1-yl)-ethanone;
1-(4-{3-[5-(1-Ethoxy-ethyl)-benzoimidazol-1-yl]-phenyl}-piperazin-1-yl)-ethanone;
1-(4-{3-[5-(1-Isopropoxy-ethyl)-benzoimidazol-1-yl]-phenyl}-piperazin-1-yl)-ethanone;
1-{4-[3-(5-Isopropyl-benzoimidazol-1-yl)-phenyl]-piperazin-1-yl}-ethanone;
   or an N-oxide thereof, any of its isomers or any mixture of its isomers,
   or a pharmaceutically acceptable salt thereof.

Any combination of two or more of the embodiments as described above is considered within the scope of the present invention.

### DEFINITION OF SUBSTITUENTS

In the context of this invention halo represents fluoro, chloro, bromo or iodo.

In the context of this invention an alkyl group designates a univalent saturated, straight or branched hydrocarbon chain. The hydrocarbon chain preferably contain of from one to six carbon atoms (C₁₋₆-alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention an alkenyl group designates a carbon chain containing one or more double bonds, including di-enes, tri-enes and poly-enes. In a preferred embodiment the alkenyl group of the invention comprises of from two to six carbon atoms (C₂₋₆-alkenyl), including at least one double bond. In a most preferred embodiment the alkenyl group of the invention is ethenyl; 1- or 2-propenyl; 1-, 2- or 3-butenyl, or 1,3-butadienyl; 1-, 2-, 3-, 4- or 5-hexenyl, or 1,3-hexadienyl, or 1,3,5-hexatrienyl.

In the context of this invention an alkynyl group designates a carbon chain containing one or more triple bonds, including di-ynes, tri-ynes and poly-ynes. In a preferred embodiment the alkynyl group of the invention comprises of from two to six carbon atoms (C₂₋₆-alkynyl), including at least one triple bond. In its most preferred embodiment the alkynyl group of the invention is ethynyl; 1-, or 2-propynyl; 1-, 2-, or 3-butynyl, or 1,3-butadiynyl; 1-, 2-, 3-, 4-pentynyl, or 1,3-pentadiynyl; 1-, 2-, 3-, 4-, or 5-henynyl, or 1,3-hexadiynyl or 1,3,5-hexatriynyl.

In the context of this invention a cycloalkyl group designates a cyclic alkyl group, preferably containing of from three to seven carbon atoms (C₃₋₇-cycloalkyl), including cyclopropyl, cyclobutyl, cyclopentyl, cyclohexyl and cycloheptyl.

Alkoxy means O-alkyl, wherein alkyl is as defined above.

Alkoxyalkyl means alkoxy as above and alkyl as above, meaning for example, methoxymethyl.

### Pharmaceutically Acceptable Salts

The chemical compound of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Examples of pharmaceutically acceptable cationic salts of a chemical compound of the invention include, without limitation, the sodium, the potassium, the calcium, the magnesium, the zinc, the aluminium, the lithium, the choline, the lysinium, and the ammonium salt, and the like, of a chemical compound of the invention containing an anionic group. Such cationic salts may be formed by procedures well known and described in the art.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Examples of pre- or prodrug forms of the chemical compound of the invention include examples of suitable prodrugs of the substances according to the invention include compounds modified at one or more reactive or derivatizable groups of the parent compound. Of particular interest are compounds modified at a carboxyl group, a hydroxyl group, or an amino group. Examples of suitable derivatives are esters or amides.

The chemical compound of the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

### Steric Isomers

It will be appreciated by those skilled in the art that the compounds of the present invention may contain one or more chiral centres and that such compounds may exist in different stereoisomeric forms—including enantiomers, diastereomers and cis-trans-isomers.

The invention includes all such isomers and any mixtures thereof including racemic mixtures.

Methods for the resolvation of optical isomers, known to those skilled in the art may be used, and will be apparent to the average worker skilled in the art. Such methods include those discussed by J. Jaques, A. Collet, and S. Wilen in “Enantiomers, Racemates, and Resolutions”, John Wiley and Sons, New York (1981).

Optical active compounds can also be prepared from optical active starting materials.

### N-Oxides

In the context of this invention an N-oxide designates an oxide derivative of a nitrogen containing compound, e.g. N-containing heterocyclic compounds capable of forming such N-oxides, and compounds holding one or more amino groups. For example, the N-oxide of a compound containing a pyridyl may be the 1-oxy-pyridin-2, -3 or -4-yl derivative.

N-oxides of the compounds of the invention may be prepared by oxidation of the corresponding nitrogen base using a conventional oxidizing agent such as hydrogen peroxide in the presence of an acid such as acetic acid at an elevated temperature, or by reaction with a peracid such as peracetic acid in a suitable solvent, e.g. dichloromethane, ethyl acetate or methyl acetate, or in chloroform or dichloromethane with 3-chloroperoxybenzoic acid.

### Labelled Compounds

The compounds of the invention may be used in their labelled or unlabelled form. In the context of this invention the labelled compound has one or more atoms replaced by an atom having an atomic mass or mass number different from the atomic mass or mass number usually found in nature. The labelling will allow easy quantitative detection of said compound.

The labelled compounds of the invention may be useful as diagnostic tools, radio tracers, or monitoring agents in various diagnostic methods, and for in vivo receptor imaging.

The labelled isomer of the invention preferably contains at least one radionuclide as a label. Positron emitting radionuclides are all candidates for usage. In the context of this invention the radionuclide is preferably selected from ²H (deuterium), ³H (tritium), ¹³C, ¹⁴C, ¹³¹I, ¹²⁵I, ¹²³I, and ¹⁸F.

The physical method for detecting the labelled isomer of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

### Methods of Preparation

The chemical compounds of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

The compounds of this invention may exist in unsolvated as well as in solvated forms with pharmaceutically acceptable solvents such as water, ethanol and the like. In general, the solvated forms are considered equivalent to the unsolvated forms for the purposes of this invention.

### Biological Activity

Compounds of the invention are capable of modulating the GABA_{A} receptor complex. They may be tested for their ability to bind to the GABA_{A} receptor complex, including specific subunits thereof.

The compounds of the present invention, being ligands for the benzodiazepine binding site on GABA_{A} receptors, are therefore of use in the treatment and/or prevention of a variety of disorders of the central nervous system. Thus in further aspect, the compounds of the invention are considered useful for the treatment, prevention or alleviation of a disease, disorder or condition responsive to modulation of the GABA_{A} receptor complex in the central nervous system.

In a special embodiment, the compounds of the invention are considered useful for the treatment, prevention or alleviation of
anxiety disorders, such as panic disorder with or without agoraphobia, agoraphobia without history of panic disorder, animal and other phobias including social phobias, obsessive-compulsive disorder, and generalized or substance-induced anxiety disorder;
stress disorders including post-traumatic and acute stress disorder;
sleep disorders;
memory disorder;
neuroses;
convulsive disorders, for example epilepsy, seizures, convulsions, or febrile convulsions in children;
migraine;
mood disorders;
depressive or bipolar disorders, for example depression, single-episode or recurrent major depressive disorder, dysthymic disorder, bipolar disorder, bipolar I and bipolar II manic disorders, and cyclothymic disorder,
psychotic disorders, including schizophrenia;
neurodegeneration arising from cerebral ischemia;
attention deficit hyperactivity disorder;
pain and nociception, e.g. neuropathic pain;
emesis, including acute, delayed and anticipatory emesis, in particular emesis induced by chemotherapy or radiation;
motion sickness, post-operative nausea and vomiting;
eating disorders including anorexia nervosa and bulimia nervosa;
premenstrual syndrome;
neuralgia, e.g. trigeminal neuralgia;
muscle spasm or spasticity, e.g. in paraplegic patients;
the effects of substance abuse or dependency, including alcohol withdrawal;
cognitive disorders, such as Alzheimer's disease;
cerebral ischemia, stroke, head trauma;
tinnitus: and
disorders of circadian rhythm, e.g. in subjects suffering from the effects of jet lag or shift work.

Preferably the compounds of the invention are considered useful for the treatment, prevention or alleviation of anxiety disorders, such as panic disorder with or without agoraphobia, agoraphobia without history of panic disorder, animal and other phobias including social phobias, obsessive-compulsive disorder, and generalized or substance-induced anxiety disorder;

Further, the compounds of the invention may be useful as radioligands in assays for detecting compounds capable of binding to the human GABA_{A} receptor.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

Preferred compounds of the invention show a biological activity in the sub-micromolar and micromolar range, i.e. of from below 1 to about 100 μM.

### Pharmaceutical Compositions

In another aspect the invention provides novel pharmaceutical compositions comprising a therapeutically effective amount of the chemical compound of the invention.

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the chemical compound of the invention, or a pharmaceutically acceptable salt or derivative thereof, together with one or more pharmaceutically acceptable carriers, and, optionally, other therapeutic and/or prophylactic ingredients, known and used in the art. The carrier(s) must be “acceptable” in the sense of being compatible with the other ingredients of the formulation and not harmful to the recipient thereof.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

A therapeutically effective dose refers to that amount of active ingredient, which ameliorates the symptoms or condition. Therapeutic efficacy and toxicity, e.g. ED₅₀ and LD₅₀, may be determined by standard pharmacological procedures in cell cultures or experimental animals. The dose ratio between therapeutic and toxic effects is the therapeutic index and may be expressed by the ratio LD₅₀/ED₅₀. Pharmaceutical compositions exhibiting large therapeutic indexes are preferred.

The dose administered must of course be carefully adjusted to the age, weight and condition of the individual being treated, as well as the route of administration, dosage form and regimen, and the result desired, and the exact dosage should of course be determined by the practitioner.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

In another aspect the invention provides a method for the treatment, prevention or alleviation of a disease or a disorder or a condition of a living animal body, including a human, which disease, disorder or condition is responsive to modulation of the GABA_{A} receptor complex in the central nervous system, and which method comprises administering to such a living animal body, including a human, in need thereof an effective amount of a chemical compound of the invention.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

### EXAMPLES

The invention is further illustrated with reference to the following examples, which are not intended to be in any way limiting to the scope of the invention as claimed.

General: All reactions involving air sensitive reagents or intermediates were performed under nitrogen and in anhydrous solvents. Magnesium sulphate or sodium sulphate was used as drying agent in the workup-procedures and solvents were evaporated under reduced pressure.

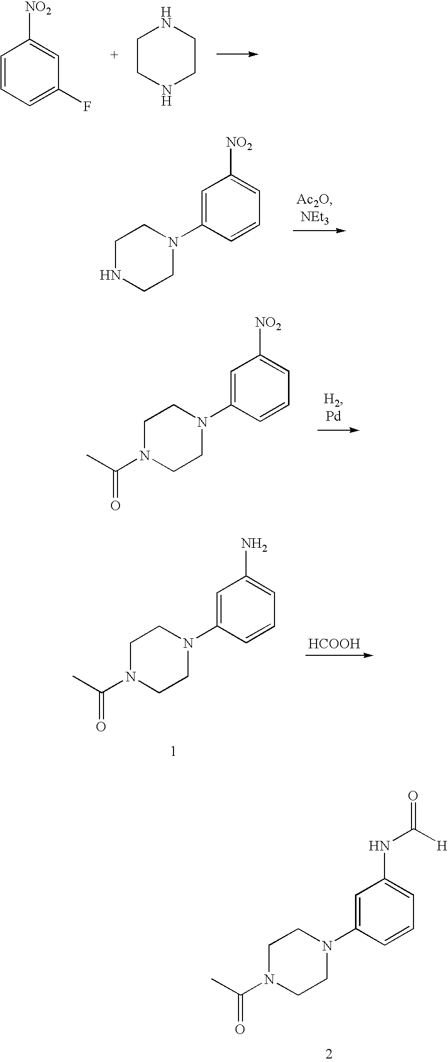

### 1-[4-(3-Aminophenyl)-piperazin-1-yl]-ethanone (1)

A suspension of 1-fluoro-3-nitrobenzene (54 ml, 0.50 mol) and piperazine (129 g, 1.5 mol) in dimethoxyethane (100 ml) was stirred at reflux under nitrogen for two days. After cooling, the solvent was removed in vacuo and the residue was partitioned between water (600 ml) and ethyl acetate (two times 400 ml). The combined organic layers were washed with brine, dried over magnesium sulfate and filtered. The filtrate was diluted with ethyl acetate to a final volume of 1500 ml and 1-(3-nitrophenyl)-piperazine, hydro chloride precipitated upon addition of ethanolic hydrogen chloride (170 ml, 3M). The precipitate was filtered off, washed with ethyl acetate and dried in the air to yield 103.3 g (85%).

To a suspension of 1-(3-nitrophenyl)-piperazine, hydro chloride (22.0 g, 0.1 mol) in acetic anhydride (150 ml) was added triethylamine (14 ml) and the resultant mixture was stirred at ambient conditions over night and then concentrated in vacuo. The concentrate was diluted with water, rendered alkaline by addition of saturated, aqueous sodium carbonate and extracted with ethyl acetate. 1-[4-(3-Nitrophenyl)-piperazin-1-yl]-ethanone was quantitatively isolated from the extract by evaporation of solvent. This intermediate was dissolved in a mixture of ethanol (150 ml) and water (50 ml). Palladium (4.8 g, 5% Pd on carbon, 50% water w/w) was added and the resultant mixture was hydrogenated at ambient pressure until the hydrogen uptake had ceased. Filtration through celite and evaporation of solvent left 1, quantitatively.

### N-[3-(4-Acetyl-piperazin-1-yl)-phenyl]-formamide (2)

A mixture of 1 (5.4 g, 24.7 mmol) and formic acid (50 ml) was stirred at reflux for 45 min. Formic acid was removed in vacuo, and the residue was partitioned between saturated, aqueous sodium carbonate and ethyl acetate. The organic layer was dried over sodium sulfate and evaporated to leave 2 (5.3 g, 87%).

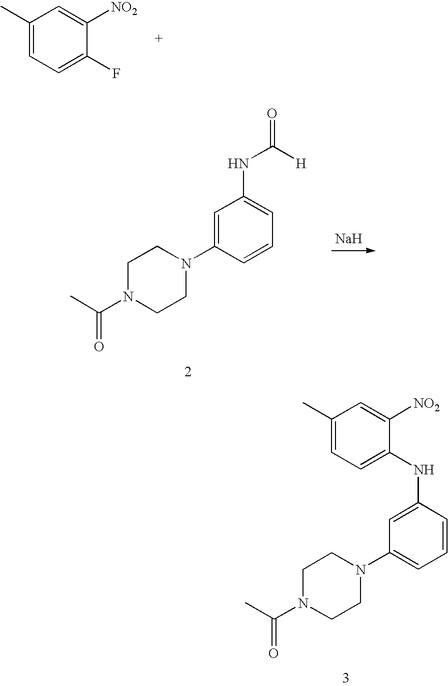

### 1-{4-[3-(4-Methyl-2-nitro-phenylamino)-phenyl]-piperazin-1-yl}-ethanone (3)

To a stirred solution of 2 (5.3 g, 21.5 mmol) in N,N-dimethyl formamide (50 ml) was added sodium hydride (1.03 g, 25.7 mmol). When the evolution of hydrogen had ceased, 4-fluoro-3-nitrotoluene (2.64 ml, 25.7 mmol) was added and the resultant mixture was stirred at 75° C. over night. The cooled mixture was poured into ice-water and extracted with ethyl acetate. The extract was dried over sodium sulfate and evaporated to dryness to afford 3, quantitatively.

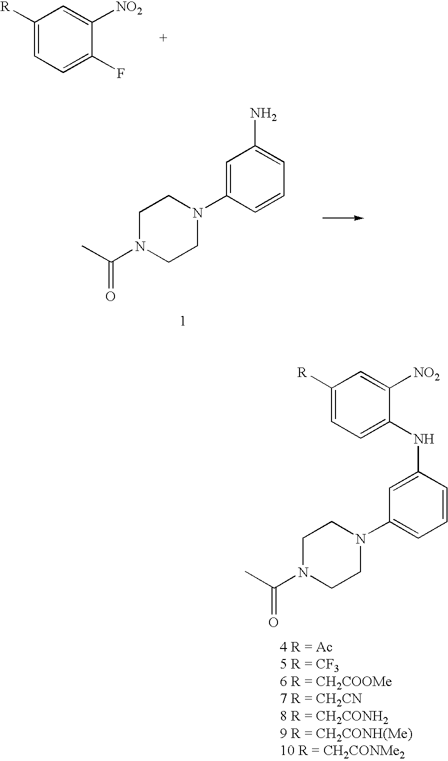

### 1-{4-[3-(4-Acetyl-piperazin-1-yl)-phenylamino]-3-nitro-phenyl}-ethanone (4)

To a solution of 4-fluoro-3-nitroacetophenone (3.34 g, 18.3 mmol) in N-methyl-2-pyrrolidinone (5 ml) was added 1 (4.0 g, 18.3 mmol) and triethylamine (2.6 ml, 18.3 mmol). The resultant mixture was stirred at 80° C. for four hours and then poured into ice-water. Extraction with dichloromethane followed by standard work-up of the extract afforded 4 (6.1 g, 87%).

### 1-{4-[3-(2-Nitro-4-trifluoromethyl-phenylamino)-phenyl]-piperazin-1-yl}-ethanone (5)

This was prepared analogously from 4-fluoro-3-nitrobenzotrifluoride in 47% yield.

### {4-[3-(4-Acetyl-piperazin-1-yl)-phenylamino]-3-nitro-phenyl}-acetic acid methyl ester (6)

This was prepared analogously from methyl (4-fluoro-3-nitrophenyl)acetate in 100% yield

as were

### {4-[3-(4-Acetyl-piperazin-1-yl)-phenylamino]-3-nitro-phenyl}-acetonitrile (7)

### {4-[3-(4-Acetyl-piperazin-1-yl)-phenylamino]-3-nitro-phenyl}-acetamide (8)

### N-Methyl {4-[3-(4-acetyl-piperazin-1-yl)-phenylamino]-3-nitro-phenyl}-acetamide (9)

### N,N-Dimethyl {4-[3-(4-acetyl-piperazin-1-yl)-phenylamino]-3-nitro-phenyl}-acetamide (10)

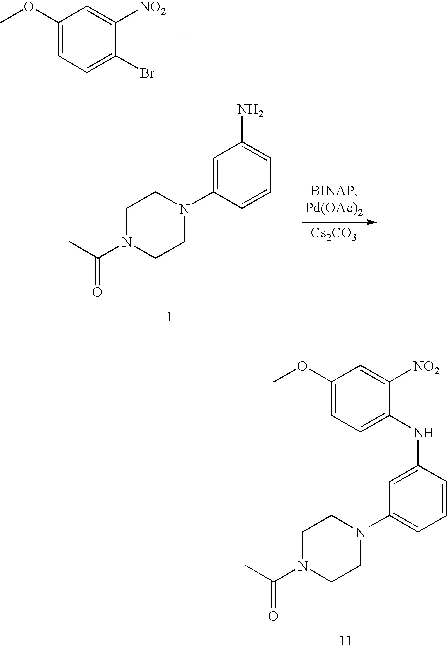

### 1-{4-[3-(4-Methoxy-2-nitro-phenylamino)-phenyl]-piperazin-1-yl}-ethanone (11)

A mixture of 4-bromo-3-nitroanisole (0.7 g, 3.0 mmol), 1 (0.66 g, 3.0 mmol), 2,2′-bis(diphenylphosphino)-1,1′binaphthyl (95 mg), palladium acetate (17 mg) and caesium carbonate (1.37 g, 4.2 mmol) in toluene (20 ml) was stirred at reflux under nitrogen for two hours. The cooled mixture was diluted with ethyl acetate and washed with water. Work-up of the organic layer by standard procedures afforded 11, quantitatively.

### 1-{4-[3-(4-Trifluoromethoxy-2-nitro-phenylamino)-phenyl]-piperazin-1-yl}-ethanone (12)

This is prepared analogously.

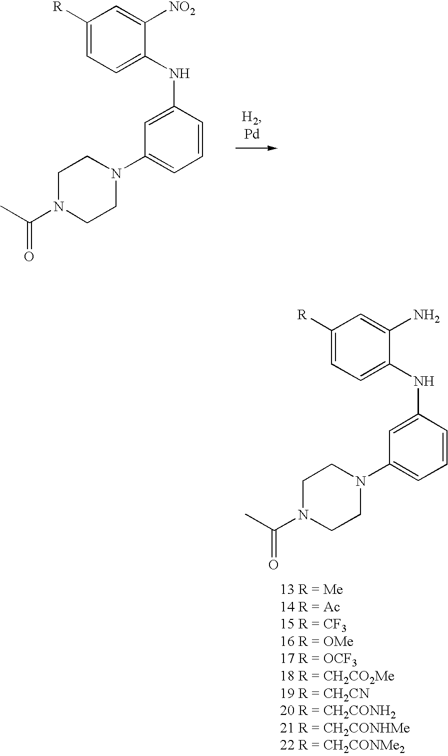

### 1-{4-[3-(2-Amino-4-methyl-phenylamino)-phenyl]-piperazin-1-yl}-ethanone (13)

To a solution of 3 (10.0 g, 28.2 mmol) in tetrahydrofurane (100 ml) was added palladium (0.5 g, 5% Pd on activated carbon) and the resultant mixture was hydrogenated until the hydrogen uptake had ceased. Filtration through celite and evaporation of solvent from the filtrate left the product, quantitatively.

### 1-{4-[3-(4-Acetyl-piperazin-1-yl)-phenylamino]-3-amino-phenyl}-ethanone (14)

This was prepared analogously from 4.

### 1-{4-[3-(2-Amino-4-trifluoromethyl-phenylamino)-phenyl]-piperazin-1-yl}-ethanone (15)

This was prepared analogously from 5.

### 1-{4-[3-(2-Amino-4-methoxy-phenylamino)-phenyl]-piperazin-1-yl}-ethanone (16)

This was prepared analogously from 11.

### 1-{4-[3-(2-Amino-4-trifluoromethoxy-phenylamino)-phenyl]-piperazin-1-yl}-ethanone (17)

This was prepared analogously from 12.

### {4-[3-(4-Acetyl-piperazin-1-yl)-phenylamino]-3-amino-phenyl}-acetic acid methyl ester (18)

This was prepared analogously from 6.

### {4-[3-(4-Acetyl-piperazin-1-yl)-phenylamino]-3-amino-phenyl}-acetonitrile (19)

This was prepared analogously from 7.

### 2-{4-[3-(4-Acetyl-piperazin-1-yl)-phenylamino]-3-amino-phenyl}-acetamide (20)

This was prepared analogously from 8.

### 2-{4-[3-(4-Acetyl-piperazin-1-yl)-phenylamino]-3-amino-phenyl}-N-methyl-acetamide (21)

This was prepared analogously from 9.

### 2-{4-[3-(4-Acetyl-piperazin-1-yl)-phenylamino]-3-amino-phenyl}-N,N-dimethyl-acetamide (22)

This was prepared analogously from 10.

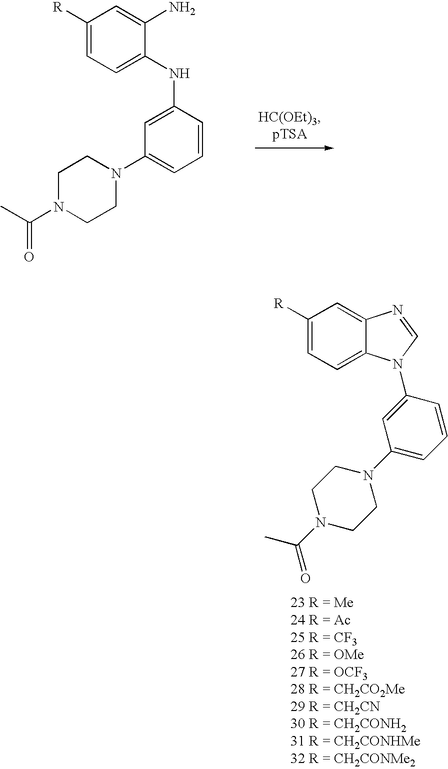

### 1-{4-[3-(5-Methyl-benzoimidazol-1-yl)-phenyl]-piperazin-1-yl}-ethanone (23)

To a solution of 13 (7.0 g, 21 mmol) in tetrahydrofurane was added three equivalents of triethyl orthoformate and a catalytic amount of p-toluenesulfonic acid. The resultant mixture was stirred at reflux for two hours whereafter the solvent was removed in vacuo. The residue was partitioned between ethyl acetate and saturated, aqueous sodium bicarbonate and the product was isolated from the organic layer by column chromatographic work-up. This afforded 23 (3.54 g, 50.5%). Mp 145-146° C.

### 1-{1-[3-(4-Acetyl-piperazin-1-yl)-phenyl]-1H-benzoimidazol-5-yl}-ethanone (24)

This was prepared analogously from 14. Yield: 89%

### 1-{4-[3-(5-Trifluoromethyl-benzoimidazol-1-yl)-phenyl]-piperazin-1-yl}-ethanone (25)

This was prepared analogously from 15. Yield: 100%. Mp 143-146° C.

### 1-{4-[3-(5-Methoxy-benzoimidazol-1-yl)-phenyl]-piperazin-1-yl}-ethanone (26)

This was prepared analogously from 16. Yield: 83%. Mp 157° C.

### 1-{4-[3-(5-Trifluoromethoxy-benzoimidazol-1-yl)-phenyl]-piperazin-1-yl}-ethanone (27)

This is prepared analogously from 17.

### {1-[3-(4-Acetyl-piperazin-1-yl)-phenyl]-1H-benzoimidazol-5-yl}-acetic acid methyl ester (28)

This was prepared analogously from 18. Yield: 31% (Isolated as the hydro chloride). m/z 393.2 (M+H)⁺

### {1-[3-(4-Acetyl-piperazin-1-yl)-phenyl]-1H-benzoimidazol-5-yl}-acetonitrile (29)

This is prepared analogously from 19.

### 2-{1-[3-(4-Acetyl-piperazin-1-yl)-phenyl]-1H-benzoimidazol-5-yl}-acetamide (30)

This is prepared analogously from 20.

### 2-{1-[3-(4-Acetyl-piperazin-1-yl)-phenyl]-1H-benzoimidazol-5-yl}-N-methyl-acetamide (31)

This is prepared analogously from 21.

### 2-{1-[3-(4-Acetyl-piperazin-1-yl)-phenyl]-1H-benzoimidazol-5-yl}-N,N-dimethyl-acetamide (32)

This is prepared analogously from 22.

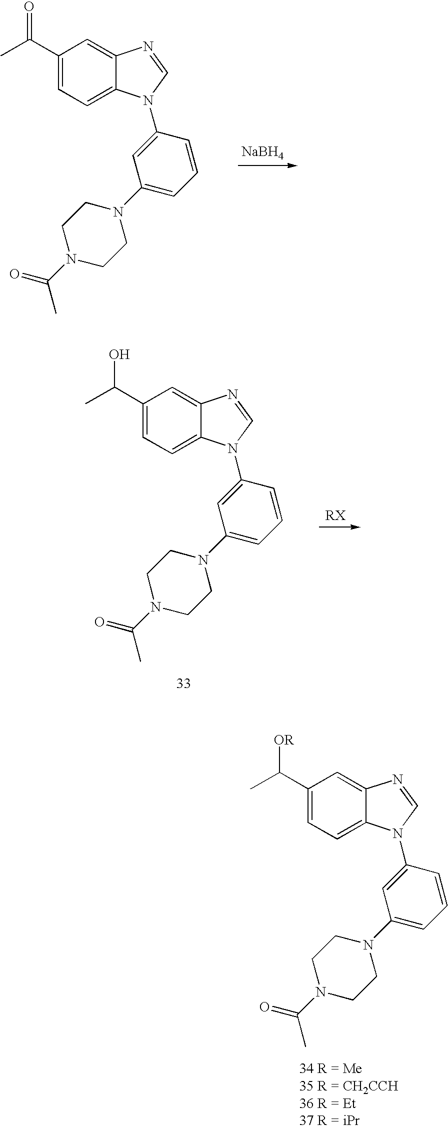

### 1-(4-{3-[5-(1-Hydroxy-ethyl)-benzoimidazol-1-yl]-phenyl}-piperazin-1-yl)-ethanone (33)

To a solution of 24 (1.65 g, 4.55 mmol) in methanol (30 ml) was added sodium boron hydride (0.17 g, 4.55 mmol) in portions over 10 min. The resultant mixture was stirred at room temperature for 1 hour and was then quenched with 4M hydrochloric acid. The mixture was rendered alkaline by addition of saturated, aqueous sodium carbonate and the product was isolated by extraction (ethyl acetate) and subsequent evaporation of solvent. Yield: 1.65 g (99%). Mp 126° C.

### 1-(4-{3-[5-(1-Methoxy-ethyl)-benzoimidazol-1-yl]-phenyl}-piperazin-1-yl)-ethanone (34), Hydro Chloride

To a stirred solution of 33 (0.5 g, 1.4 mmol) in N,N-dimethyl formamide (10 ml) was added sodium hydride (60 mg, 60% dispersion in mineral oil). When the evolution of hydrogen had ceased, iodomethane (0.2 ml, 3 mmol) was added and stirring was continued at room temperature over night. The reaction mixture was poured into water and extracted with ethyl acetate. The extract was dried over sodium sulfate and concentrated in vacuo. The residue was dissolved in a small volume of tetrahydrofurane, and the product was precipitated as the hydrochloride by addition of etheral hydrogen chloride. Yield: 0.4 g (70%). m/z 379.2 (M+H)⁺

### 1-(4-{3-[5-(1-Prop-2-ynyloxy-ethyl)-benzoimidazol-1-yl]-phenyl}-piperazin-1-yl)-ethanone (35), Hydrochloride

This was prepared analogously from 33 and propargyl bromide. Yield 52%. m/z 403.2 (M+H)⁺

### 1-(4-{3-[5-(1-Ethoxy-ethyl)-benzoimidazol-1-yl]-phenyl}-piperazin-1-yl)-ethanone (36)

This is prepared analogously from 33 and iodoethane.

### 1-(4-{3-[5-(1-Isopropoxy-ethyl)-benzoimidazol-1-yl]-phenyl}-piperazin-1-yl)-ethanone (37)

This is prepared analogously from 33 and 2-bromopropane.

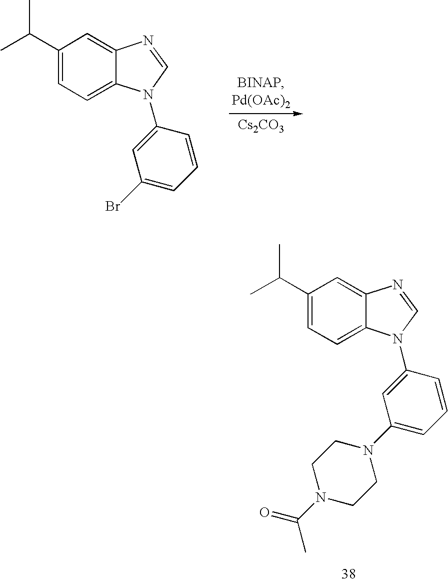

### 1-{4-[3-(5-Isopropyl-benzoimidazol-1-yl)-phenyl]-piperazin-1-yl}-ethanone (38), Hydro Chloride

A mixture of 1-(3-bromo-phenyl)-5-isopropyl-1H-benzoimidazole (1.5 g, 5 mmol), N-acetyl piperazine (0.64 g, 5 mmol), 2,2′-bis(diphenylphosphino)-1,1′binaphthyl (0.16 g), palladium acetate (28 mg) and caesium carbonate (2.3 g, 7 mmol) in toluene (40 ml) was stirred at reflux under nitrogen over night. The cooled reaction mixture was diluted with ethyl acetate and washed with water. The solvent was removed in vacuo and the residue was purified by column chromatography on silica gel, eluting with a mixture of ethyl acetate and methanol (9:1, v/v). The product precipitated as the hydrochloride from the pure fractions upon addition of etheral hydrogen chloride. Yield: 0.76 g (42%). m/z 363.2 (M+H)⁺

### Test Methods

### In Vitro Inhibition of ³H-flunitrazepam (³H-FNM) Binding

The GABA recognition site and the benzodiazepine modulatory unit can selectively be labelled with ³H-flunitrazepam.

### Tissue Preparation

Preparations are performed at 0-4° C. unless otherwise indicated. Cerebral cortex from male Wistar rats (150-200 g) is homogenised for 5-10 sec in 20 ml Tris-HCl (30 mM, pH 7.4) using an Ultra-Turrax homogeniser. The suspension is centrifuged at 27,000×g for 15 min and the pellet is washed three times with buffer (centrifuged at 27,000×g for 10 min). The washed pellet is homogenized in 20 ml of buffer and incubated on a water bath (37° C.) for 30 min to remove endogenous GABA and then centrifuged for 10 min at 27,000×g. The pellet is then homogenized in buffer and centrifuged for 10 min at 27,000×g. The final pellet is resuspended in 30 ml buffer and the preparation is frozen and stored at −20° C.

### Assay

The membrane preparation is thawed and centrifuged at 2° C. for 10 min at 27,000×g. The pellet is washed twice with 20 ml 50 mM Tris-citrate, pH 7.1 using an Ultra-Turrax homogeniser and centrifuged for 10 min at 27,000×g. The final pellet is resuspended in 50 mM Tris-citrate, pH 7.1 (500 ml buffer per g of original tissue), and then used for binding assays. Aliquots of 0.5 ml tissue are added to 25 μl of test solution and 25 μl of ³H-FNM (1 nM, final concentration), mixed and incubated for 40 min at 2° C. Non-specific binding is determined using Clonazepam (1 μM, final concentration). After incubation the samples are added 5 ml of ice-cold buffer and poured directly onto Whatman GF/C glass fibre filters under suction and immediately washed with 5 ml ice-cold buffer. The amount of radioactivity on the filters is determined by conventional liquid scintillation counting. Specific binding is total binding minus non-specific binding.

### Results

25-75% inhibition of specific binding must be obtained, before calculation of an IC₅₀.

The test value will be given as IC₅₀ (the concentration (μM) of the test substance which inhibits the specific binding of ³H-FNM by 50%).

$${IC}_{50} = ( applied test substance concentration , µM ) \times \frac{1}{( \frac{{C}_{o}}{{C}_{x}} - 1 )}$$

where

Cₒ is specific binding in control assays, and

Cₓ is the specific binding in the test assay.

(The calculations assume normal mass-action kinetics).

Test results from these experiments with a number of compounds of the invention are shown in Table 1 below.

TABLE 1

| | In vitro binding |
|---|---|
| Test compound | IC₅₀ (μM) |
| Compound 23 | 0.0026 |
| Compound 33 | 0.0096 |
| Compound 38 | 0.0050 |

1-13. (canceled)

14. A compound of the general formula (I): 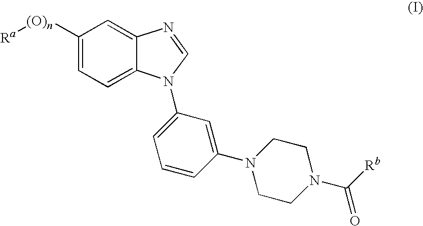
or an N-oxide thereof, any of its stereoisomers or any mixture of its stereoisomers,
or a pharmaceutically acceptable salt thereof,
wherein
R^{a} represents an alkyl group;
which alkyl group is optionally substituted with one or more substituents independently selected from the group consisting of:
halo, hydroxy, R′R″N—, cyano, nitro, cycloalkyl, alkenyl, alkynyl, alkoxy, alkenyloxy, alkynyloxy, formyl, alkylcarbonyl, alkoxycarbonyl, alkoxyalkylcarbonyl or R′R″N-carbonyl-;
wherein R′ and R″ independent of each other are hydrogen or alkyl;
n is 0 or 1;
R^{b} represents hydrogen or alkyl.

15. The compound of claim 14, or a pharmaceutically acceptable salt thereof, wherein R^{a} represents alkyl.

16. The compound of claim 14, or a pharmaceutically acceptable salt thereof, wherein R^{a} represents perhaloalkyl.

17. The compound of claim 14, or a pharmaceutically acceptable salt thereof, wherein R^{a} represents alkyl substituted with cyano, hydroxyl, alkoxy, alkenyloxy, alkynyloxy, alkoxycarbonyl, or R′R″N-carbonyl-.

18. The compound of claim 14, or a pharmaceutically acceptable salt thereof, wherein n is 0.

19. The compound of claim 14, or a pharmaceutically acceptable salt thereof, wherein n is 1.

20. The compound of claim 14, or a pharmaceutically acceptable salt thereof, wherein R^{b} represents alkyl.

21. The compound of claim 14, which is
1-{4-[3-(5-Methyl-benzoimidazol-1-yl)-phenyl]-piperazin-1-yl}-ethanone;
1-{4-[3-(5-Trifluoromethyl-benzoimidazol-1-yl)-phenyl]-piperazin-1-yl}-ethanone;
1-{4-[3-(5-Methoxy-benzoimidazol-1-yl)-phenyl]-piperazin-1-yl}-ethanone;
1-{4-[3-(5-Trifluoromethoxy-benzoimidazol-1-yl)-phenyl]-piperazin-1-yl}-ethanone;
{1-[3-(4-Acetyl-piperazin-1-yl)-phenyl]-1H-benzoimidazol-5-yl}-acetic acid methyl ester;
{1-[3-(4-Acetyl-piperazin-1-yl)-phenyl]-1H-benzoimidazol-5-yl}-acetonitrile;
2-{1-[3-(4-Acetyl-piperazin-1-yl)-phenyl]-1H-benzoimidazol-5-yl}-acetamide;
2-{1-[3-(4-Acetyl-piperazin-1-yl)-phenyl]-1H-benzoimidazol-5-yl}-N-methyl-acetamide;
2-{1-[3-(4-Acetyl-piperazin-1-yl)-phenyl]-1H-benzoimidazol-5-yl}-N,N-dimethyl-acetamide;
1-(4-{3-[5-(1-Hydroxy-ethyl)-benzoimidazol-1-yl]-phenyl}-piperazin-1-yl)-ethanone;
1-(4-{3-[5-(1-Methoxy-ethyl)-benzoimidazol-1-yl]-phenyl}-piperazin-1-yl)-ethanone;
1-(4-{3-[5-(1-Prop-2-ynyloxy-ethyl)-benzoimidazol-1-yl]-phenyl}-piperazin-1-yl)-ethanone;
1-(4-{3-[5-(1-Ethoxy-ethyl)-benzoimidazol-1-yl]-phenyl}-piperazin-1-yl)-ethanone;
1-(4-{3-[5-(1-Isopropoxy-ethyl)-benzoimidazol-1-yl]-phenyl}-piperazin-1-yl)-ethanone;
1-{4-[3-(5-Isopropyl-benzoimidazol-1-yl)-phenyl]-piperazin-1-yl}-ethanone;
or an N-oxide thereof, any of its isomers or any mixture of its isomers,
or a pharmaceutically acceptable salt thereof.

22. A pharmaceutical composition, comprising a therapeutically effective amount of a compound of claim 14, or an N-oxide thereof, any of its stereoisomers or any mixture of its stereoisomers, or a pharmaceutically acceptable salt thereof, together with at least one pharmaceutically acceptable carrier, excipient or diluent.

23. A method for treatment, prevention or alleviation of a disease or a disorder or a condition of a living animal body, including a human, which disorder, disease or condition is responsive to modulation of the GABA_{A} receptor complex in the central nervous system, which method comprises the step of administering to such a living animal body in need thereof a therapeutically effective amount of a compound according to claim 14, or an N-oxide thereof, any of its stereoisomers or any mixture of its stereoisomers, or a pharmaceutically acceptable salt thereof.

24. The method according to claim 23, for the manufacture of a pharmaceutical composition for the treatment, prevention or alleviation of a disease or a disorder or a condition of a mammal, including a human, which disease, disorder or condition is responsive to modulation of the GABA_{A} receptor complex in the central nervous system.

25. The method according to claim 24, wherein the disease, disorder or condition is anxiety disorders, panic disorder with or without agoraphobia, agoraphobia without history of panic disorder, animal and other phobias, social phobias, obsessive-compulsive disorder, and generalized or substance-induced anxiety disorder; stress disorders, post-traumatic and acute stress disorder, sleep disorders, memory disorder, neuroses, convulsive disorders, epilepsy, seizures, convulsions, febrile convulsions in children, migraine, mood disorders, depressive or bipolar disorders, depression, single-episode or recurrent major depressive disorder, dysthymic disorder, bipolar disorder, bipolar I and bipolar II manic disorders, cyclothymic disorder, psychotic disorders, including schizophrenia, neurodegeneration arising from cerebral ischemia, attention deficit hyperactivity disorder, pain, nociception, neuropathic pain, emesis, acute, delayed and anticipatory emesis, particular emesis induced by chemotherapy or radiation, motion sickness, post-operative nausea, vomiting, eating disorders, anorexia nervosa, bulimia nervosa, premenstrual syndrome, neuralgia, trigeminal neuralgia, muscle spasm, spasticity, e.g. in paraplegic patients, the effects of substance abuse or dependency, alcohol withdrawal, cognitive disorders, Alzheimer's disease, cerebral ischemia, stroke, head trauma, tinnitus or disorders of circadian rhythm, e.g. in subjects suffering from the effects of jet lag or shift work.
